(19) United States
(12) Patent Application Publication
(10) Pub. No.: US 2023/0255565 A1
(43) Pub. Date: Aug. 17, 2023

(54) INFORMATION PROCESSING DEVICE, INFORMATION PROCESSING METHOD, AND INFORMATION PROCESSING PROGRAM

(71) Applicant: FUJIFILM CORPORATION, Tokyo (JP)

(72) Inventors: Haruyasu NAKATSUGAWA, Kanagawa (JP); Yasuhisa KANEKO, Kanagawa (JP); Kenji NAGAMIYA, Kanagawa (JP); Tomohide HIRAGAMI, Kanagawa (JP); Yasuyuki HOSONO, Kanagawa (JP); Nobuya KITAMURA, Kanagawa (JP); Keiji TSUBOTA, Kanagawa (JP)

(21) Appl. No.: 18/304,372

(22) Filed: Apr. 21, 2023

Related U.S. Application Data
(63) Continuation of application No. PCT/JP2021/039889, filed on Oct. 28, 2021.

(30) Foreign Application Priority Data
Nov. 9, 2020 (JP) 2020-186802

(51) Int. Cl. A61B 5/00

(52) U.S. Cl. CPC A61B 5/7275; A61B 5/746; A61B 5/6824

(57) ABSTRACT

An information processing device comprising at least one processor, wherein the at least one processor is configured to recommend, as a method of inputting control information for controlling a device to be controlled to a control device that controls the device to be controlled, a noncontact input method of inputting the control information in a manner noncontact with the control device in a case where a predetermined condition is satisfied.

### CROSS REFERENCE TO RELATED APPLICATIONS

This application is a continuation of International Application No. PCT/JP2021/039889, filed on Oct. 28, 2021, which claims priority from Japanese Patent Application No. 2020-186802, filed on Nov. 9, 2020. The entire disclosure of each of the above applications is incorporated herein by reference.

### BACKGROUND

### Technical Field

The present disclosure relates to an information processing device, an information processing method, and an information processing program.

### Related Art

Hitherto, in a case where a plurality of operators handle a control device that controls medical equipment, a technique for reducing a risk of spread of an infectious disease through the control device due to coming into contact with the control device, to which a pathogen is attached, is disclosed.

For example, JP2010-042207A describes that, in a case where a person other than a predetermined operator comes into contact or approaches an operation panel, control is performed such that an operation button is not disposed in a contact or approaching region. For example, JP2013-180207A discloses that an ultrasound probe is operated by a combination of an input by a noncontact input device, such as voice and gesture, and an input by a contact input device, such as a mouse and a keyboard.

In recent years, from a viewpoint of reducing a risk of infection with an infectious disease, there is an increasing demand for control of medical equipment by a noncontact input method, such as voice and gesture, by which infection through the control device is difficult to occur. On the other hand, for example, in a situation in which infection through the control device is difficult to occur, such as a case where only a specific operator handles the control device, there is a demand for enabling reception of control of medical equipment by a contact input method, such as a mouse, a keyboard, a button, and a touch panel.

### SUMMARY

The present disclosure provides an information processing device, an information processing method, and an information processing program capable of recommending an input method depending on a situation.

According to a first aspect of the present disclosure, there is provided an information processing device comprising at least one processor, in which the processor is configured to recommend, as a method of inputting control information for controlling a device to be controlled to a control device that controls the device to be controlled, a noncontact input method of inputting the control information in a manner noncontact with the control device in a case where a predetermined condition is satisfied.

According to a second aspect of the present disclosure, in the aspect, the condition may be a predetermined condition as a condition that a risk of infection with an infectious disease due to contact with the control device increases.

According to a third aspect of the present disclosure, in the aspect, the processor may be configured to recommend an input of the control information by the noncontact input method in a case where the control device is shared.

According to a fourth aspect of the present disclosure, in the aspect, the device to be controlled may be at least one of an image capturing device, a sample examination device, or a physiological examination device, and the condition may be a condition regarding an examination order pertaining to the device to be controlled.

According to a fifth aspect of the present disclosure, in the fourth aspect, the processor may be configured to recommend an input of the control information by the noncontact input method in a case where designation is made in the examination order that a sample is under a suspicion of contamination or infection.

According to a sixth aspect of the present disclosure, in the fourth or fifth aspect, the processor may be configured to recommend an input of the control information by the noncontact input method in a case where a round by the device to be controlled is designated in the examination order.

According to a seventh aspect of the present disclosure, in the fourth to sixth aspects, the processor may be configured to recommend an input of the control information by the noncontact input method in a case where an input by the noncontact input method is designated in the examination order.

According to an eighth aspect of the present disclosure, in the fourth to seventh aspects, the processor may be configured to recommend an input of the control information by the noncontact input method in a case where an examination method designated in the examination order is a predetermined method as an examination method at a high risk of infection.

According to a ninth aspect of the present disclosure, in the fourth to eighth aspects, the processor may be configured to recommend an input of the control information by the noncontact input method in a case where a type of a sample designated in the examination order is a predetermined type as a type of a sample at a high risk of infection.

According to a tenth aspect of the present disclosure, in the aspect, the condition may be a condition regarding a state of presence of a person in a predetermined region where the control device is used.

According to an eleventh aspect of the present disclosure, in the tenth aspect, the processor may be configured to recommend an input of the control information by the noncontact input method in a case where at least one of the number of people who are present in the region or the total number of hours for which a person is present in the region is equal to or greater than a predetermined threshold value.

According to a twelfth aspect of the present disclosure, in the tenth or eleventh aspect, the processor may be configured to recommend an input of the control information by the noncontact input method in a case where a contaminated or infected person is present in the region.

According to a thirteenth aspect of the present disclosure, in the aspect, the processor may be configured to recommend an input of the control information by the noncontact input method in a case where a present point of time corresponds to a predetermined period.

According to a fourteenth aspect of the present disclosure, in the aspect, the processor may be configured to issue a warning in a case where an input of the control information by the noncontact input method is recommended, and the input of the control information is executed by a method different from the noncontact input method.

According to a fifteenth aspect of the present disclosure, in the aspect, the processor may be configured to determine whether or not the condition is satisfied.

According to a sixteenth aspect of the present disclosure, there is provided an information processing method by which a computer executes a process comprising recommending, as a method of inputting control information for controlling a device to be controlled to a control device that controls the device to be controlled, a noncontact input method of inputting the control information in a manner noncontact with the control device in a case where a predetermined condition is satisfied.

According to a seventeenth aspect of the present disclosure, there is provided an information processing program that causes a computer to execute a process comprising recommending, as a method of inputting control information for controlling a device to be controlled to a control device that controls the device to be controlled, a noncontact input method of inputting the control information in a manner noncontact with the control device in a case where a predetermined condition is satisfied.

According to the aspect, the information processing device, the information processing method, and the information processing program of the present disclosure can recommend an input method depending on a situation.

### BRIEF DESCRIPTION OF THE DRAWINGS

FIG. 1 is a schematic configuration diagram of an information processing system.

FIG. 2 is a block diagram showing an example of a hardware configuration of an information processing device.

FIG. 3 is a block diagram showing an example of a functional configuration of the information processing device.

FIG. 4 is a diagram showing an example of an examination order.

FIG. 5 is a diagram showing an example of a screen that is displayed on a display.

FIG. 6 is a diagram showing an example of a screen that is displayed on the display.

FIG. 7 is a flowchart illustrating an example of information processing.

FIG. 8 is a diagram showing an example of a wearing form of a wearable terminal using a connecting member.

### DETAILED DESCRIPTION

Hereinafter, an embodiment for realizing the technique of the present disclosure will be described in detail referring to the drawings.

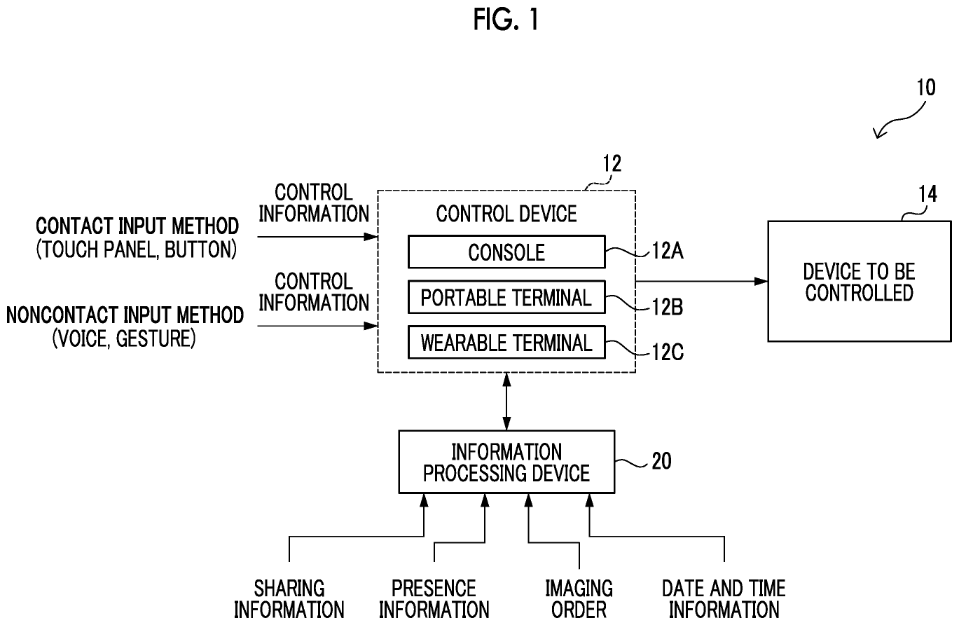

Referring to FIG. 1, an example of a configuration of an information processing system 10 according to the present exemplary embodiment will be described. As shown in FIG. 1, the information processing system 10 comprises a control device 12, a device 14 to be controlled and an information processing device 20. The information processing device 20 and the control device 12, and the control device 12 and the device 14 to be controlled can perform communication with each other through a network. In the present exemplary embodiment, an example where the control device 12, the device 14 to be controlled, and the information processing device 20 are provided in a hospital will be described.

The control device 12 is a device that receives control information for controlling the device 14 to be controlled, input by an operator and controls the device 14 to be controlled depending on the control information. As the control device 12, for example, a console 12A that includes a computer, a portable terminal 12B, such as a smartphone and a tablet, and a wearable terminal 12C, such as a wrist watch type, an eyeglasses type, an earphone type, and a ring type, can be applied. Hereinafter, such devices are referred to as the “control device 12” in a case where there is no distinction therebetween.

The device 14 to be controlled is a device that is controlled by the control device 12, and is, for example, at least one of an image capturing device, a sample examination device, or a physiological examination device. The image capturing device is, for example, a device that captures an image of a sample by a method, such as computed tomography (CT), digital radiography (DR), computed radiography (CR), angiography, magnetic resonance imaging (MRI), and ultrasonic waves. The sample examination device is, for example, a device that is used to perform various examinations, such as component analysis and detection of a pathogen using a sample, such as urine, feces, blood, nasopharyngeal swab, paranasal swab, saliva, and other body fluids. The physiological examination device is, for example, a device that is used to measure and examine a physiological function of a subject, such as cardiac electricity, cardiac sound, myoelectricity, pulse waves, brain waves, audibility, and a respiratory function.

As shown in FIG. 1, the control device 12 receives an input of control information by two kinds of methods of a contact input method by which an operator comes into contact with the control device 12 to input the control information and a noncontact input method by which the operator inputs the control information in a manner noncontact with the control device 12. Various input devices that are used for the operator to input the control information are provided or connected in or to the control device 12.

The contact input method is specifically a method that inputs the control information to the control device 12 using various input devices accompanied with contact in an input of the control information, such as a keyboard, a mouse, a touch panel, a touch sensor, and a button. Examples of the contact input method include an input of the control information using a keyboard and a mouse connected to the console 12A and an input of the control information using a touch panel of the portable terminal 12B.

The noncontact input method is specifically a method that inputs the control information to the control device 12 without contact with the device in an input of the control information, such as an voice input, a gesture input, a brain wave input and a sight line input, and an input using a noncontact panel on which an input can be performed in a state in which a finger is floated from the panel. Examples of the noncontact input method include a gesture input using a sensor in the wearable terminal 12C, a voice input using a microphone in the portable terminal 12B, and a gesture input using a camera in the console 12A. It is premised that the wearable terminal 12C is worn on a body of the operator, and contact in a case where the operator wears the wearable terminal 12C is permitted even in the “noncontact input method”.

As a method of the voice input, the gesture input, the brain wave input, or the sight line input, a known method can be appropriately applied. For example, the gesture input may be performed by detecting movement of a body, such as a finger, a hand, or an arm, of the operator with an accelerator sensor, a gyro sensor, an optical sensor, a tactile sensor, or a smart textile (see U.S. Ser. No. 10/488,936B, US2019/0004604A, US2014/0194742A). For example, the gesture input may be performed by detecting movement of the body of the operator through analysis on a video obtained by imaging the operator with a camera. For example, the brain wave input may be performed by detecting brain waves of the operator with electrodes on the scalp of the operator (see U.S. Pat. No. 9,864,431B).

The information processing device 20 according to the present exemplary embodiment has a function of recommending the noncontact input method in a case where it is estimated that the operator who handles the control device 12 is at a high risk of infection with an infectious disease through the control device 12. This is because that the noncontact input method by which infection through the control device 12 is difficult to occur is superior from a viewpoint of reducing the risk of infection with the infectious disease. On the other hand, in a situation in which infection through the control device 12 is difficult to occur, such as a case where only a specific operator handles the control device 12, the input method is not limited to the noncontact input method, and reception of an input of the control information by the contact input method is preferably enabled. Hereinafter, an example of the configuration of the information processing device 20 according to the present exemplary embodiment will be described.

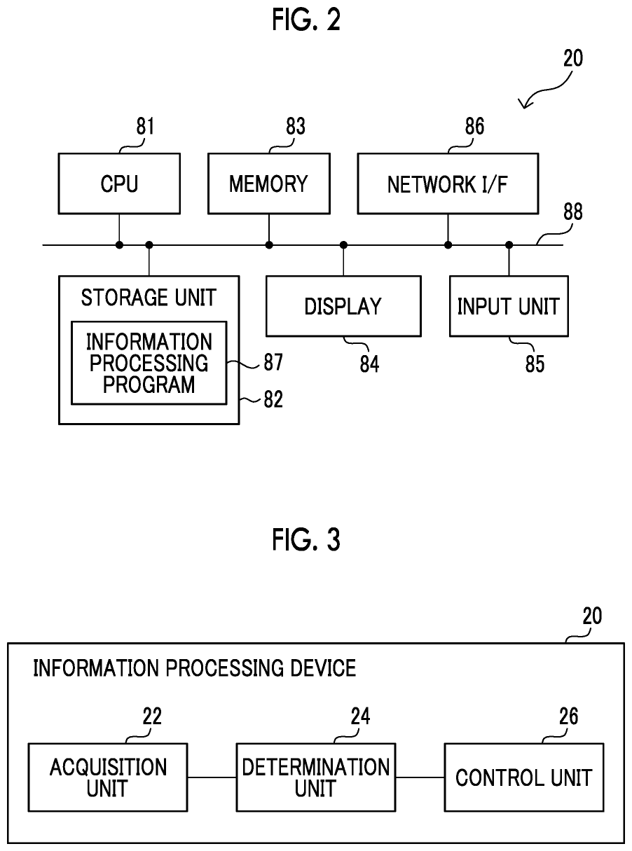

First, the hardware configuration of the information processing device 20 according to the present exemplary embodiment will be described referring to FIG. 2. As shown in FIG. 2, the information processing device 20 includes a central processing unit (CPU) 81, a non-volatile storage unit 82, and a memory 83 as a temporary storage area. The information processing device 20 includes a display 84, such as a liquid crystal display, an input unit 85, such as a keyboard and a mouse, and a network interface (I/F) 86 that is connected to a network. The CPU 81, the storage unit 82, the memory 83, the display 84, the input unit 85, and the network I/F 86 are connected to be able to transfer various kinds of information with each other through a bus 88, such as a system bus and a control bus.

The storage unit 82 is realized by a storage medium, such as a hard disk drive (HDD), a solid state drive (SSD), and a flash memory. An information processing program 87 is stored in the storage unit 82. The CPU 81 reads out the information processing program 87 from the storage unit 82, develops the information processing program 87 to the memory 83, and executes the developed information processing program 87. The CPU 81 is an example of a processor of the present disclosure.

Next, the functional configuration of the information processing device 20 according to the present exemplary embodiment will be described referring to FIG. 3. As shown in FIG. 3, the information processing device 20 includes an acquisition unit 22, a determination unit 24, and a control unit 26. The CPU 81 functions as the acquisition unit 22, the determination unit 24, and the control unit 26 by executing the information processing program 87.

The acquisition unit 22 acquires various kinds of information that are used to determine whether or not the operator who handles the control device 12 is at a high risk of infection with an infectious disease through the control device 12. Specifically, the acquisition unit 22 acquires at least one of information (hereinafter, referred to as “sharing information”) indicating a state of sharing of the control device 12, information (hereinafter, referred to as “presence information”) indicating a state of presence of a person in a predetermined region where the control device 12 is used, an examination order pertaining on imaging and examination by the device 14 to be controlled, or date and time information indicating date and time of a present point of time.

Here, “sharing” is not limited to a case where a plurality of operators handle the control device 12 side by side, and may include, for example, a case where a different operator handles the control device 12 before cleaning of the control device 12 is performed after the control device 12 is handled by a certain operator.

The sharing information may be input, for example, by the operator through the input unit 85. For example, the sharing information may be information that is generated based on a log obtained in a case where the operator who handles the control device 12 causes an integrated circuit (IC) tag reader provided for each control device 12 to read an IC tag carried with the operator at the acquisition unit 22 or an external device. For example, the sharing information may be information that is generated by imaging the control device 12 with a camera and executing image analysis processing on a captured video at the acquisition unit 22 or the external device.

Here, the “predetermined region where the control device 12 is used” is, for example, a control room where the control device 12 is disposed. The presence information specifically includes information indicating at least one of the number of people who are present in the predetermined region where the control device 12 is used or the total number of hours for which a person is present. The “total number of hours” is a time obtained by accumulating a time from entrance to exit of one person in a region for all people who enter the region. A person that is a target to be included in the presence information is not limited to the operator of the control device 12, and may include, for example, a subject of imaging or examination by the device 14 to be controlled, a companion of the subject, and a medical employee who works at the hospital.

The presence information may be input, for example, by the operator through the input unit 85. For example, the present information may be information that is generated based on a log obtained in a case where a person who enters a region causes an IC tag reader provided for each predetermined region where the control device 12 is used, to read an IC tag carried with the person at the acquisition unit 22 or an external device. For example, the presence information may be information that is generated by imaging the predetermined region where the control device 12 is used, with a camera and executing image analysis processing on a captured video at the acquisition unit 22 or the external device. For example, the presence information may be information that is generated based on position information obtained using a portable terminal, a wearable terminal, a tag, or the like carried with a person. As a measurement method of the position information, a known method using Bluetooth (Registered Trademark), a wireless local area network (LAN), a global positioning system (GPS), an ultrasonic wave, or the like can be applied.

The presence information may include information indicating a state of infection or contamination of a person who is present in the predetermined region where the control device 12 is used. Specifically, the presence information may include information indicating whether or not a person who is suspected of infection or contamination, such as an infected person of an infectious disease or a person who has close contact to the infected person is present in the predetermined region where the control device 12 is used. Information indicating the state of infection or contamination of a person may be input, for example, by the operator through the input unit 85. For example, the presence information may be information that is generated based on an examination result, a heartbeat and a body temperature acquired by the wearable terminal 12C worn on each person, or the like at the acquisition unit 22 or the external device.

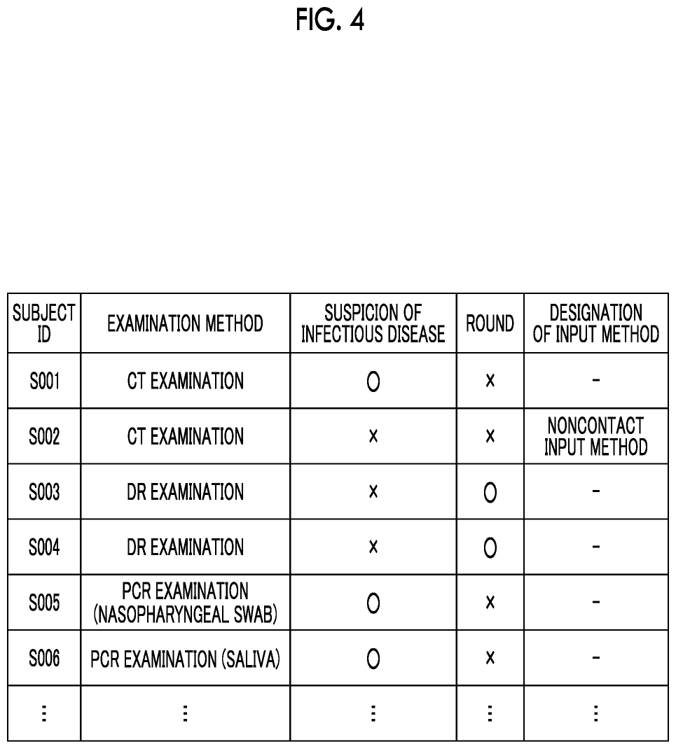

The examination order is acquired, for example, from a management device that performs unified management of examination contents by the device 14 to be controlled. FIG. 4 shows an example of the examination order. As shown in FIG. 4, identification information (“subject identification (ID)” of FIG. 4) of a subject and an examination method are included in the examination order. Furthermore, in the examination order, information (“suspicion of infectious disease” of FIG. 4) indicating whether or not there is a suspicion of contamination or infection in the subject, information (“round” of FIG. 4) indicating whether or not an examination is performed in a round, and information for designating an input method are included. In a field of “suspicion of infectious disease” of FIG. 4, a circle mark is added in a case where the subject is suspected of contamination or infection, and a cross mark is added in a case where the subject is not suspected of contamination or infection. In a field of “round” of FIG. 4, a circle mark is added in a case where an examination is performed in a round, and a cross mark is added in a case where an examination is not performed in a round.

The determination unit 24 determines whether or not the predetermined condition is satisfied, based on various kinds of information acquired by the acquisition unit 22. The “condition” is specifically a predetermined condition as a condition that a risk of infection with an infectious disease due to coming into contact with the control device 12 increases, and is stored, for example, in the storage unit 82 in advance. Hereinafter, the condition will be described.

First, the determination unit 24 may determine that the condition is satisfied in a case where the control device 12 is shared, based on the sharing information acquired by the acquisition unit 22. This is because, in a case where the control device 12 is shared, and a pathogen is attached to the control device 12, it is considered that a risk of infection of the operator through the control device 12 increases.

Second, the determination unit 24 may determine whether or not various conditions regarding a state of presence of a person in the predetermined region where the control device 12 is used are satisfied, based on the presence information acquired by the acquisition unit 22. Specifically, the determination unit 24 may determine that the condition is satisfied in a case where at least one of the number of people who are present in a region or the total number of hours for which a person is present in a region, indicated by the presence information is equal to or greater than a predetermined threshold value. This is because it is considered that, as the number of people who enter the region is large and as the total number of hours is long, a pathogen is attached to the control device 12 with a person who enters the region, as a generation source, and a risk of infection of the operator who comes into contact with the control device 12 increases.

Specifically, the determination unit 24 may determine that the condition is satisfied in a case where the presence information indicates that a contaminated or infected person is present in the predetermined region where the control device 12 is used. This is because, in a case where a contaminated or infected person is present in the predetermined region where the control device 12 is used, a pathogen is attached to the control device 12 with the person as a generation source, and a risk of infection of the operator who comes into contact with the control device 12 increases.

Third, the determination unit 24 may determine whether or not a condition regarding the examination order pertaining to the device 14 to be controlled is satisfied, based on the examination order acquired by the acquisition unit 22. Specifically, the determination unit 24 may determine that the condition is satisfied in a case where designation is made in the examination order that a sample is under a suspicion of contamination or infection. In the example of FIG. 4, examination orders of the “subject ID” 5001, 5005, and 5006 correspond to such a case. This is because there is a case where a sample that is a target of examination by the device 14 to be controlled approaches the control device 12, a pathogen is attached to the control device 12 with the sample as a generation source, and a risk of infection of the operator who comes into contact with the control device 12 increases.

Specifically, the determination unit 24 may determine that the condition is satisfied in a case where a round by the device 14 to be controlled is designated in the examination order. In the example of FIG. 4, examination orders of the “subject ID” 5003 and 5004 correspond to such a case. This is because, in a case where a round is performed, a lot of contaminated or infected samples approach the control device 12 compared to a case where a round is not performed, it is considered that there is a high possibility that a pathogen is attached to the control device 12, and a risk of infection of the operator who comes into contact with the control device 12 increases.

Specifically, the determination unit 24 may determine that the condition is satisfied in a case where an input by the noncontact input method is designated in the examination order. In the example of FIG. 4, an examination order of the “subject ID” 5002 corresponds to such a case. For example, in a case where it is known in advance that an examination is at a high risk of contamination or infection, the noncontact input method is designated in the examination order in advance, whereby it is possible to reliably recommend an input by the noncontact input method. For example, in a case where the noncontact input method is preferably used depending on examination contents, the noncontact input method can also be designated.

Specifically, the determination unit 24 may determine that the condition is satisfied in a case where an examination method designated in the examination order is a predetermined method as an examination method at a high risk of infection. An example of the examination method at a high risk of infection is polymerase chain reaction (PCR) examination intended for detecting whether or not a subject is infected with a pathogen, such as a novel coronavirus (COVID-19) and an influenza virus. It is considered that the PCR examination is an examination method by which a sample is at a high risk of contamination or infection, a pathogen is attached to the control device 12 with the sample as a generation source, and a risk of infection of the operator who comes into contact with the control device 12 increases. Such a “predetermined examination method as an examination method at a high risk of infection” is stored, for example, in the storage unit 82 in advance.

Specifically, the determination unit 24 may determine that the condition is satisfied in a case where a type of a sample designated in an examination order is a predetermined type as a type of a sample at a high risk of infection. For example, in the PCR examination, nasopharyngeal swab, paranasal swab, and saliva can be used as a sample. In a case where nasopharyngeal swab is used as a sample in the PCR examination, it is considered that a subject sneezes in collecting nasopharyngeal swab from an inner part of a nose of the subject, and a pathogen is attached to the control device 12 in the surroundings of the subject, so that a risk of infection of the operator who comes into contact with the control device 12 increases. On the other hand, in a case where saliva is used as a sample in the PCR examination, since the subject does not sneeze unlike a case where nasopharyngeal swab is used, it is possible to restrain a pathogen from being attached to the control device 12 in the surroundings of the subject, and a risk of infection of the operator who comes into contact with the control device 12 is reduced. In this way, in a case where a risk of infection is different depending on the type of the sample that is used in the examination, it is preferable that determination is made whether or not the condition is satisfied, depending on the type of the sample. Such a “predetermined type as a type of a sample at a high risk of infection” is stored, for example, in the storage unit 82 in advance.

Fourth, the determination unit 24 may determine that the condition is satisfied in a case where date and time of a present point of time indicated date and time information acquired by the acquisition unit 22 corresponds to a predetermined period. As the “predetermined period”, for example, a period during which prevalence of an infectious disease is known in advance from statistics, such as winter that is a prevalence period of an influenza virus infectious disease in Japan, can be applied. This is because, in a case where the present point of time corresponds to such a period, it is considered that there is a high possibility that a contaminated or infected sample approaches the control device 12 and a pathogen is attached to the control device 12, compared to a case where the present point of time does not correspond to such a period, a risk of infection of the operator who comes into contact with the control device 12 increases. Information indicating the “predetermined period” is stored, for example, in the storage unit 82 in advance.

The determination unit 24 may perform determination by appropriately combining the respective conditions described above. For example, in a case where the present point of time corresponds to the predetermined period, the determination unit 24 may perform a reduction of the threshold value that is used in the determination of the number of people who are present in the region and the total number of hours indicated by the presence information (that is, facilitate the recommendation of the noncontact input method), or the like.

The control unit 26 performs control for recommending the noncontact input method as a method of inputting the control information to the control device 12 in a case where the determination unit 24 determines that the predetermined condition is satisfied. For example, the control unit 26 may perform control for displaying a screen D with a content for recommending the noncontact input method on a display device, such as a liquid crystal display and an organic electro luminescence (EL) display. The display device may be provided, for example, in the information processing device 20, the control device 12, and the device 14 to be controlled, or may be separately provided at a place where the operator can view the screen.

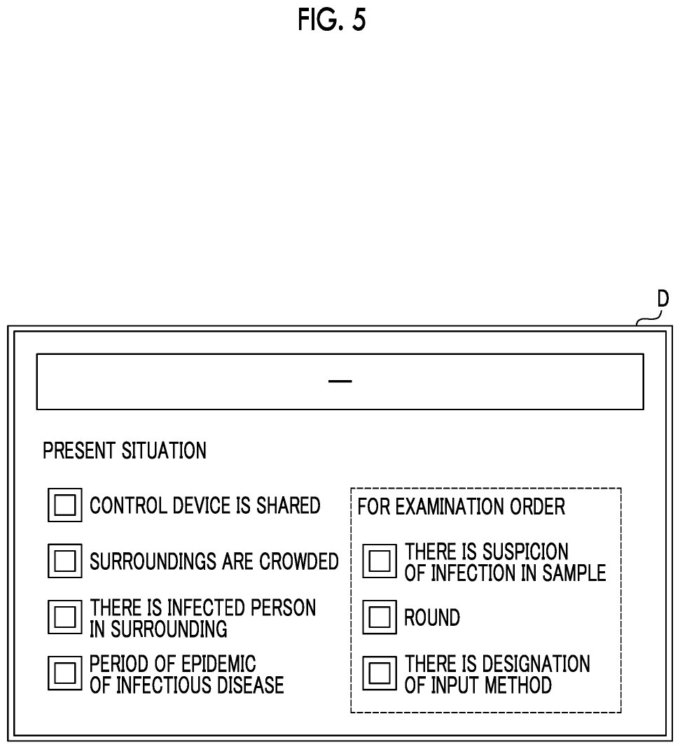

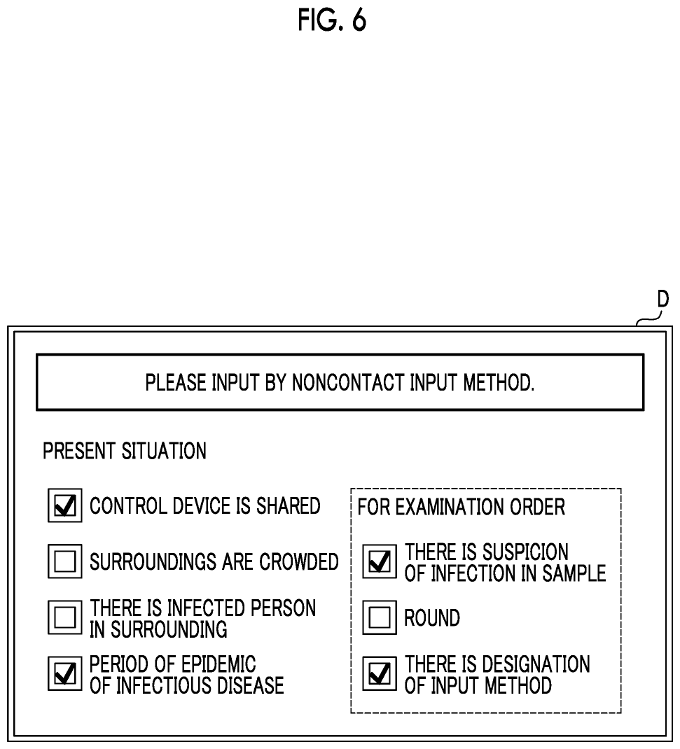

FIGS. 5 and 6 show an example of the screen D that is displayed on the display device by the control unit 26. FIG. 5 shows a screen D in an initial state (that is, in a case where the noncontact input method is not particularly recommended). FIG. 6 shows a screen D in a case where the noncontact input method is recommended. As shown in FIG. 6, the control unit 26 performs control such that a screen with a content for recommending the noncontact input method, such as “Please input by noncontact input method”, is displayed on the display device in a case where the determination unit 24 determines that the predetermined condition is satisfied.

As shown in FIGS. 5 and 6, the control unit 26 may perform control for displaying the screen D that includes various conditions and information indicating whether or not various conditions are satisfied, on the display device. In the example of FIG. 6, a check mark is added to various conditions determined that the condition is satisfied, by the determination unit 24.

The control unit 26 may perform control such that the noncontact input method is recommended, for example, by a notification device that can notify of information by lighting of a light emitting diode (LED) lamp, ringing of a buzzer, an announcement, or the like. The notification device may be provided, for example, in the information processing device 20, the control device 12, and the device 14 to be controlled or may be separately provided at a place where the operator can recognize the notification.

The control unit 26 may recommend the noncontact input method in a case where even any one of various predetermined conditions is satisfied or may recommend only in a case where all conditions are satisfied. The control unit 26 may recommend the noncontact input method in a case where only a predetermined number of conditions among various predetermined conditions are satisfied.

The control unit 26 may perform control for issuing a warning in a case where the input of the control information by the noncontact input method is recommended, and the input of the control information is executed by a method (that is, the contact input method) different from the noncontact input method. Specifically, the control unit 26 may perform control for issuing a warning with a content, such as “There is no recommended input method.”, by the display device and the notification device.

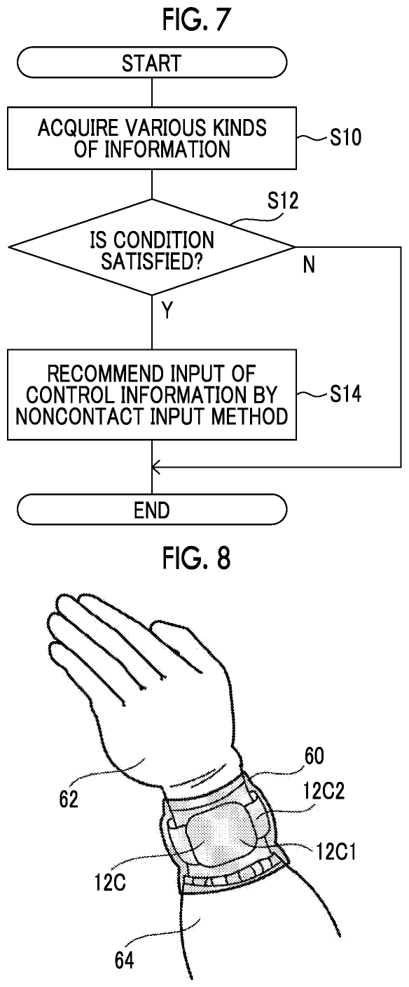

Next, the operations of the information processing device 20 according to the present exemplary embodiment will be described referring to FIG. 7. The CPU 81 executes the information processing program 87, whereby information processing shown in FIG. 7 is executed. The information processing shown in FIG. 7 is executed, for example, for each examination by the device 14 to be controlled.

In Step S10 of FIG. 7, the acquisition unit 22 acquires at least one of various kinds of information of the sharing information, the presence information, the examination order, and the date and time information. In Step S12, the determination unit 24 determines whether or not the predetermined condition is satisfied, based on various kinds of information acquired in Step S10. In a case where affirmative determination is made in Step S12, the process proceeds to Step S14, and the control unit 26 performs control for recommending the input of the control information by the noncontact input method. In a case where Step S14 ends and in a case where negative determination is made in Step S12, the control unit 26 ends the process.

As described above, the information processing device 20 according to the present exemplary embodiment comprises at least one processor, and the processor recommends the noncontact input method of inputting the control information in a manner noncontact with the control device 12 as the method of inputting the control information for controlling the device 14 to be controlled to the control device 12 that controls the device 14 to be controlled, in a case where the predetermined condition is satisfied. Accordingly, since it is possible to recommend the input method depending on the situation, such as a case where it is estimated that the operator who handles the control device 12 is at a high risk of infection with an infectious disease, it is advantageous to suppress the spread of the infectious disease.

In the above-described exemplary embodiment, in a case where the operator comes into contact with an input device that is not shared by other operators, to input the control information, this may be permitted as a “noncontact input method”. For example, in a case where the portable terminal 12B and the wearable terminal 12C are given for each operator and are not shared, the operator may be permitted to come into contact with a touch panel or the like thereof and to input the control information. For example, in a case where the operator performs an operation of an input device that is shared by a plurality of operators, using a touch pen dedicated to the operator (that is, while coming into contact with a touch pen with which other people do not come into contact), this may be regarded as a “noncontact input method”.

In the above-described exemplary embodiment, the presence information may be determined in consideration of a protection state of a person who is present in a region. This is because it is considered that a protected person is less likely to spread an infectious disease than an unprotected person. The protection state is determined, for example, depending on a wearing state of a mask and protective clothing, and an execution state of hand washing, gargling, and disinfection with alcohol. The presence information is determined in consideration of the protection state of the person who is present in the region, whereby it is possible to appropriately suppress the spread of the infectious disease.

In this case, for example, the presence information may be generated while making a weight of the number of people different, such as counting a person wearing a mask as 0.5 persons and counting a person insufficiently washing the hands as 1.5 persons. For example, the presence information may be generated while making a weight different depending on a type of a mask, such as a non-woven fabric mask, a gauze mask, a surgical mask, and a particulate mask. The presence or absence of wearing of the mask of the person and the type of the mask can be determined based on, for example, an image obtained by imaging the person with a camera. Determination about whether or not hand washing is sufficient can be performed based on whether or not a time determined as a time during which the person is washing the hands is equal to or greater than a predetermined threshold value, for example, by a function of a wearable terminal.

In the above-described exemplary embodiment, the presence information may be set such that, in a case where a person who exits the predetermined region where the control device 12 is used returns to the same region again, the person is not counted twice as the number of people who are present in the region. This is because it is considered that the same person returns to the same region and an infectious disease is less likely to further spread. The same person is not included twice, whereby it is possible to generate the presence information more conforming to the state of each region.

In the above-described exemplary embodiment, in a case where it is detected that at least one of the number of people who are present in the predetermined region where the control device 12 is used or the total number of hours is likely to be equal to or greater than the predetermined threshold value, based on the presence information, the determination unit 24 may notify the control unit 26 of the fact. In this case, the control unit 26 may perform control such that the operator is notified of the fact by the display device and the notification device. For example, the control unit 26 may notify of a message, such as “The surroundings of the control device 12 are crowded, and then, the noncontact input method is recommended before long.” in a case where at least one of the number of people who are present in the region or the total number of hours falls within a predetermined range from the predetermined threshold value. According to such a configuration, it is possible to serve a timing at which the operator executes a method for suppressing the spread of the infectious disease, such as restriction on exit from a region and entrance to a region, isolation of a person, and execution of ventilation and cleaning, and it is advantageous to suppress the spread of the infectious disease.

In the above-described exemplary embodiment, a configuration in which a plurality of different kinds of terminals (the console 12A, the portable terminal 12B, and the wearable terminal 12C) are appropriately combined as the control device 12 may be used. For example, a configuration in which the operator performs the input of the control information with the wearable terminal 12C owned by the operator, and the wearable terminal 12C transmits the control information to the device 14 to be controlled through the portable terminal 12B or the console 12A may be used. According to such a configuration, since the wearable terminal 12C owned by an individual and the device 14 to be controlled are not directly connected, it is advantageous in security.

In the above-described exemplary embodiment, although a configuration in which the information processing system 10 is applied to the hospital has been described, the technique of the present disclosure is not limited thereto. The information processing system 10 according to the present exemplary embodiment can be applied to, for example, various facilities, such as an office, a school, an accommodation facility, an eating facility, a nursing facility, and a refugee facility.

In the above-described exemplary embodiment, although a configuration in which medical equipment is applied as the device 14 to be controlled has been described, the technique of the present disclosure is not limited thereto, and various kinds of equipment that operate in a case where the operator inputs the control information can be applied. Examples of such equipment include information equipment, such as a computer and a smartphone, communication equipment, such as a router and a firewall, household electrical appliances, such as a television and an air conditioner, and business equipment, such as a copying machine, a printer, and a scanner device.

In the above-described exemplary embodiment, although a configuration in which the determination unit 24 of the information processing device 20 determines whether or not various conditions are satisfied has been described, the technique of the present disclosure is not limited thereto. For example, a configuration in which the control device 12, the device 14 to be controlled, or any external device determines whether or not various conditions are satisfied and transmits a result of determination to the information processing device 20 through a network may be used.

In the above-described exemplary embodiment, for example, as the hardware structure of processing units that execute various kinds of processing, such as the acquisition unit 22, the determination unit 24, and the control unit 26, various processors described below can be used. Various processors include a programmable logic device (PLD) that is a processor capable of changing a circuit configuration after manufacturing, such as a field programmable gate array (FPGA), a dedicated electric circuit that is a processor having a circuit configuration dedicatedly designed for executing specific processing, such as an application specific integrated circuit (ASIC), and the like, in addition to a CPU that is a general-purpose processor that executes software (program) to function as various processing units, as described above.

One processing unit may be configured with one of various processors described above or may be configured with a combination of two or more processors (for example, a combination of a plurality of FPGAs or a combination of a CPU and an FPGA) of the same type or different types. A plurality of processing units may be configured with one processor.

As an example where a plurality of processing units are configured with one processor, first, as represented by a computer, such as a client or a server, a configuration in which one processor is configured with a combination of one or more CPUs and software, and the processor functions as a plurality of processing units is known. Second, as represented by System on Chip (SoC) or the like, a configuration in which a processor that realizes the functions of the entire system including a plurality of processing units by one integrated circuit (IC) chip is used is known. In this way, various processing units are configured using one or more processors among various processors described above as the hardware structure.

In addition, as the hardware structure of various processors is, more specifically, an electric circuit (circuitry), in which circuit elements, such as semiconductor elements, are combined can be used.

In the above-described exemplary embodiment, although an aspect where the information processing program 87 is stored (installed) in the storage unit 82 has been described, the technique of the present disclosure is not limited thereto. The information processing program 87 may be provided in a form of being recorded on a recording medium, such as a compact disc read only memory (CD-ROM), a digital versatile disc read only memory (DVD-ROM), and a universal serial bus (USB) memory. Alternatively, a configuration in which the information processing program 87 is downloaded from an external device through a network may be used. The technique of the present disclosure also extends to a storage medium that non-transitorily stores the information processing program, in addition to the information processing program.

[Wearing Method of Wrist Watch Type Wearable Terminal]

Here, a wearing method of a wrist watch type wearable terminal 12C (so-called smart watch) that can be applied as the control device 12 in the above-described exemplary embodiment will be described in connection with a preferred embodiment. Here, it is assumed that the wrist watch type wearable terminal 12C is brought into direct contact with a skin of a wearer and can acquire biological information, such as a heartbeat and a body temperature of the wearer. It is assumed that the wearer of the wearable terminal 12C puts on protective clothing. Normally, in a case where the person puts on the protective clothing, sleeves of the protective clothing are accommodated in protective gloves such that writes are not exposed. For this reason, in a case where the person puts on the protective clothing in a state of wearing the wrist watch type wearable terminal 12C on the wrist, the wearable terminal 12C is covered with the protective clothing and is not viewed.

Accordingly, in a case where the operator who puts on the protective clothing wears the wrist watch type wearable terminal 12C on the wrist, as shown in FIG. 8, a connecting member 60 that covers the exposed wrist and connects a wrist-side end of a protective glove 62 and a sleeve of protective clothing 64 is preferably used. The connecting member 60 contains, for example, transparent or semitransparent resin. According to such a configuration, it is possible to wear the wrist watch type wearable terminal 12C in a state of being brought into direct contact with the wrist without damaging the function of the protective clothing, and to enable acquisition of biological information by the wearable terminal 12C. Since the wearable terminal 12C does not come into contact with external air, it is possible to reduce a frequency and a degree of cleaning and sterile filtration work of the wearable terminal 12C, and it is advantageous to improve efficiency of work.

In the connecting member 60, a portion corresponding to a screen portion 12C1 of the wrist watch type wearable terminal 12C may be swollen compared to other portions. The connecting member 60 may be provided with a number of fine holes to an extent not to transmit a pathogen in a portion that includes at least the portion corresponding to the screen portion 12C1 of the wrist watch type wearable terminal 12C. According to such a configuration, in a case where the wearable terminal 12C has a voice input function using a microphone, the wearable terminal 12C easily acquires voice.

The above-described configuration using the connecting member 60 is an example of a configuration that can be applied to a case where the wrist watch type wearable terminal 12C is applied as the control device 12 in the above-described exemplary embodiment, and the technique of the present disclosure is not limited thereto. For example, in a case where the biological information of the wearer the wearable terminal 12C is not acquired by the wearable terminal 12C, the person may wear the wearable terminal 12C over the protective clothing. For example, in a case where a person may expose a wrist without putting on the protective clothing, the person may wear the wearable terminal 12C to be brought into direct contact with the wrist, and the connecting member 60 may not be used. In such cases, for example, a frequency and a degree of cleaning and sterile filtration work of the screen portion 12C1 of the wearable terminal 12C and infection countermeasures, such as replacement with a cleaned and sterile filtrated belt 12C2, preferably increase.

The technique of the present disclosure can also combine the above-described embodiment as appropriate. The content of the above description and the content of the drawings are detailed description of portions according to the technique of the present disclosure, and are merely examples of the technique of the present disclosure. For example, description regarding the above configurations, functions, operations, and advantages are description regarding an example of the configurations, functions, operations, and advantages of the portions according to the technique of the present disclosure. Thus, it is needless to say that unnecessary portions may be deleted, new elements may be added, or replacements may be made in the above-described contents and the illustrated contents without departing from the gist of the technique of the present disclosure.

The entire disclosure of JP2020-186802, filed Nov. 9, 2020, is incorporated into the present specification by reference. All documents, patent applications, and technical standards described in the present specification are incorporated in the specification by reference to the same extent as in a case where the individual document, patent application, or technical standard is specifically and individually indicated to be incorporated by reference.

What is claimed is:

1. An information processing device comprising at least one processor, wherein the at least one processor is configured to recommend, as a method of inputting control information for controlling a device to be controlled to a control device that controls the device to be controlled, a noncontact input method of inputting the control information in a manner noncontact with the control device in a case where a predetermined condition is satisfied.

2. The information processing device according to claim 1, wherein the condition is a predetermined condition as a condition that a risk of infection with an infectious disease due to contact with the control device increases.

3. The information processing device according to claim 1, wherein the at least one processor is configured to recommend an input of the control information by the noncontact input method in a case where the control device is shared.

4. The information processing device according to claim 1, wherein:
the device to be controlled is at least one of an image capturing device, a sample examination device, or a physiological examination device, and
the condition is a condition regarding an examination order pertaining to the device to be controlled.

5. The information processing device according to claim 4, wherein the at least one processor is configured to recommend an input of the control information by the noncontact input method in a case where designation is made in the examination order that a sample is under a suspicion of contamination or infection.

6. The information processing device according to claim 4, wherein the at least one processor is configured to recommend an input of the control information by the noncontact input method in a case where a round by the device to be controlled is designated in the examination order.

7. The information processing device according to claim 4, wherein the at least one processor is configured to recommend an input of the control information by the noncontact input method in a case where an input by the noncontact input method is designated in the examination order.

8. The information processing device according to claim 4, wherein the at least one processor is configured to recommend an input of the control information by the noncontact input method in a case where an examination method designated in the examination order is a predetermined method as an examination method at a high risk of infection.

9. The information processing device according to claim 4, wherein the at least one processor is configured to recommend an input of the control information by the noncontact input method in a case where a type of a sample designated in the examination order is a predetermined type as a type of a sample at a high risk of infection.

10. The information processing device according to claim 1, wherein the condition is a condition regarding a state of presence of a person in a predetermined region where the control device is used.

11. The information processing device according to claim 10, wherein the at least one processor is configured to recommend an input of the control information by the noncontact input method in a case where at least one of the number of people who are present in the region or the total number of hours for which a person is present in the region is equal to or greater than a predetermined threshold value.

12. The information processing device according to claim 10, wherein the at least one processor is configured to recommend an input of the control information by the noncontact input method in a case where a contaminated or infected person is present in the region.

13. The information processing device according to claim 1, wherein the at least one processor is configured to recommend an input of the control information by the noncontact input method in a case where a present point of time corresponds to a predetermined period.

14. The information processing device according to claim 1, wherein the at least one processor is configured to issue a warning in a case where an input of the control information by the noncontact input method is recommended, and the input of the control information is executed by a method different from the noncontact input method.

15. The information processing device according to claim 1, wherein the at least one processor is configured to determine whether or not the condition is satisfied.

16. An information processing method by which a computer executes a process comprising:
recommending, as a method of inputting control information for controlling a device to be controlled to a control device that controls the device to be controlled, a noncontact input method of inputting the control information in a manner noncontact with the control device in a case where a predetermined condition is satisfied.

17. A non-transitory computer-readable storage medium storing an information processing program that causes a computer to execute a process comprising:
recommending, as a method of inputting control information for controlling a device to be controlled to a control device that controls the device to be controlled, a noncontact input method of inputting the control information in a manner noncontact with the control device in a case where a predetermined condition is satisfied.
